(19) United States
(12) Patent Application Publication
(10) Pub. No.: US 2008/0188769 A1
(43) Pub. Date: Aug. 7, 2008

(54) Foldable Brush Self-sampling Device

(76) Inventor: Li-Cheng LU, Taoyuan City (TW)

Correspondence Address: Dr. BANGER SHIA, 102 Lindencrest Ct., Sugar Land, TX, 77479-5201, US

(21) Appl. No.: 11/672,145

(22) Filed: Feb. 7, 2007

(51) Int. Cl. A61B 10/02

(52) U.S. Cl. 600/569

(57) ABSTRACT

A foldable brush self-sampling device for collecting of cell specimens from a body cavity, such as cervix, endometrium, rectum, nasal cavity and fauces, for diagnostic purposes, allows a user to collect cell specimens in the privacy of home. The device includes a sheath and a brush bar. The brush bar would fold into the sheath, which is with smaller diameter. The user inserts the sheath into the cavity easily and pushes the brush bar to expose the brush elements. After insertion of the sheath and push of the brush bar, the brush bar is rotated to obtain the specimen from the body cavity and then retracted in order to return the sheath.

### BACKGROUND OF THE INVENTION

1. Field of Invention

The present invention generally relates to the collecting of cell specimens from a body cavity for diagnostic purposes and, more particularly, is concerned with a foldable brush self-sampling device for collecting cell specimens of cervix, endometrium, rectum, nasal cavity and fauces.

2. Related Art

During the 1940's, Dr. George Papanicolaou developed a screening test which bears his name and which has become the most widely used screening technique for detecting abnormal cervical cells. Today, this test is known more commonly as the PAP test or the PAP smear test. Typically, the PAP smear test is performed in the physician's office as part of a woman's routine gynecological examination. If identified early and treated, deaths of cervical cancer are completely unnecessary because up to 100% of the most common types of cervical cancer may be prevented. Compared with invasive cervical cancer which has a cure rate of 50% to 60% in average, the PAP smear test for screening cervical cancer is of great worth. In cytology, normal cervical cells are columnar cells, have medium-sized nuclear, no hyperchromatic nuclei and no mitotic figures. On the side, if cells have no clear cell border, larger and darker nuclei, increased nuclear/cytoplasmic area ratio, increased mitotic figures and a disorderly arrangement, the cells are abnormal. The clinical classification of the abnormal cells has five degrees as follows. The defined class I is normal, class II is inflammation or atrophy (like a menopause variation), class III is cervical dysplasia (between normal cells and cancer cells), class IV is pre-cancer cell (carcinoma in situ), and class V is malignancy.

Typically, the PAP test is performed by inserting a speculum into the patient's vagina to expose the cervix. The surface of the cervix is then scraped by a brush, stick or swab and the exfoliated cells thereby collected are smeared upon a microscope slide for cytological examination.

However, the conventional smear test needs to collect cervical cells firstly and that process needs to insert a collector like stick or swab into the patient's vagina and then reach to the cervix. If the collector is not inserting enough to reach to the cervix, the exfoliated cells collected have no means because of the collected cells are vagina epidermal cells if the location the collector reached is too shallow and it is easy to cause a erroneous judgment. On the contrary, the collected cells are endometrial cells if the collector is inserting too deep and the uterus also may be injured by the collector.

Furthermore, the PAP smear test is nearly always performed in a physician's office by a gynecologist or other medical professional that almost is male. Thus, the test is considered by many women to be uncomfortable and embarrassing. Therefore, a personal cervical cell collector that will allow a female patient to collect cervical cells in the privacy of her home is required.

The modem people have been bustling about life and the air pollution is to be serious. They are easier to affect carcinoma of the rectum and nasopharyngeal cancer. However, most people are disinclined to the hospital for inspection and often tow to the symptom worsens. Consequently, a need exists for a cytologic sampling device, which is convenient for people to collect cell specimens by themselves.

### SUMMARY OF THE INVENTION

The present invention provides a foldable brush self-sampling device for collecting of cell specimens from a body cavity, such as cervix, endometrium, rectum, nasal cavity and fauces, for diagnostic purposes. The device is simple in construction, employs relatively inexpensive materials, and is easy and quick to use. Also, the use of the device is relatively painless to the patient and devoid of complications to early diagnosis and treatment.

Accordingly, the present invention is directed to a foldable brush self-sampling device for collecting cell specimens from a body cavity, such as cervix, endometrium, rectum, nasal cavity and fauces, for diagnostic purposes. The foldable brush self-sampling device comprises a sheath and a brush bar mounted on the sheath, and the brush bar comprises an elongated stem and a plurality of brush elements formed in a front end portion of the stem by a base. The base is greater than the sheath and would be folded into the sheath together with the brush elements, wherein in using, the base is exposed from the sheath by pushing the stem, and the base would extend to permit collecting interior specimen of humans.

Also, for preventing to be stained with body fluid, the device further includes a cover in a front end of the sheath. And, when using in cervical cell collecting, the device would includes a tubular outer shell and a guide assembly to make sampling easily for women themselves.

It is to be understood that both the foregoing general description and the following detailed description are exemplary, and are intended to provide further explanation of the invention as claimed.

### BRIEF DESCRIPTION OF THE DRAWINGS

In the following detailed description, reference will be made to the attached drawings in which:

FIG. 1A is a plan view of a foldable brush self-sampling device in accordance with the present invention.

FIG. 1B is a plan view of a foldable brush self-sampling device in accordance with the present invention during folding.

FIG. 1C is a plan view of a foldable brush self-sampling device in accordance with the present invention when folded.

FIG. 2A is a plan view of a foldable brush self-sampling device in accordance with the present invention when using in vagina.

FIG. 2B is a plan view of a foldable brush self-sampling device in accordance with the present invention when collecting cervical cells.

FIG. 3A is a plan view of a foldable brush self-sampling device with a cover in accordance with the present invention.

FIG. 3B is a plan view of another embodiment of a foldable brush self-sampling device with a cover in accordance with the present invention.

FIG. 4A is a plan view of an outer shell connected with a guide assembly of a foldable brush self-sampling device in accordance with the present invention.

FIG. 4B is a plan view of the outer shell connected with a sampling assembly of the foldable brush self-sampling device in FIG. 4A.

FIG. 4C is a plan view of the foldable brush self-sampling device in FIG. 4B connected with a camera

FIGS. 5A to 5D are operative views of foldable brush self-sampling device in FIG. 4A.

FIG. 5E is operative views of foldable brush self-sampling device in FIG. 4B.

FIG. 6 is operative views of foldable brush self-sampling device in FIG. 4C.

### DETAILED DESCRIPTION OF THE INVENTION

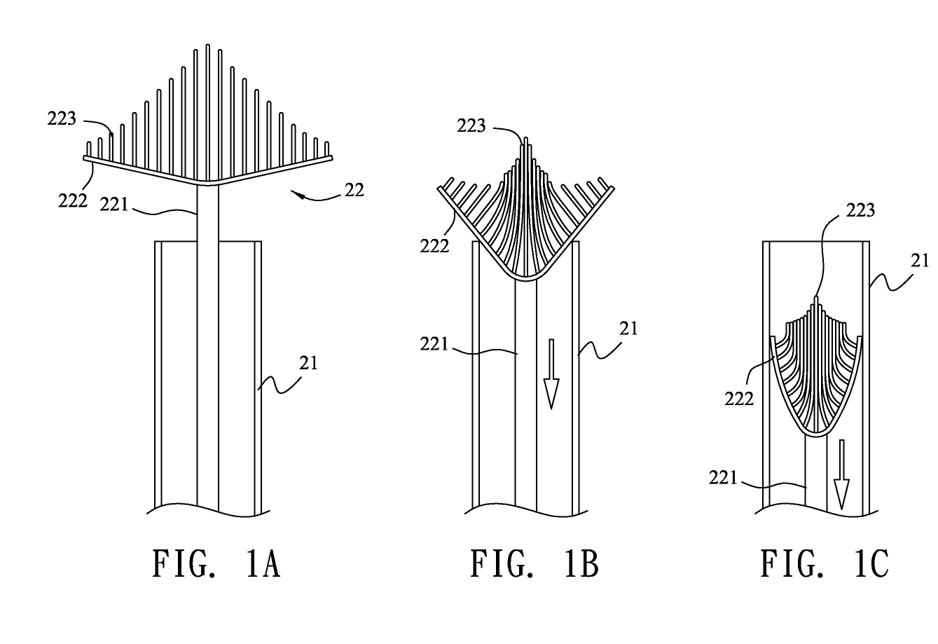

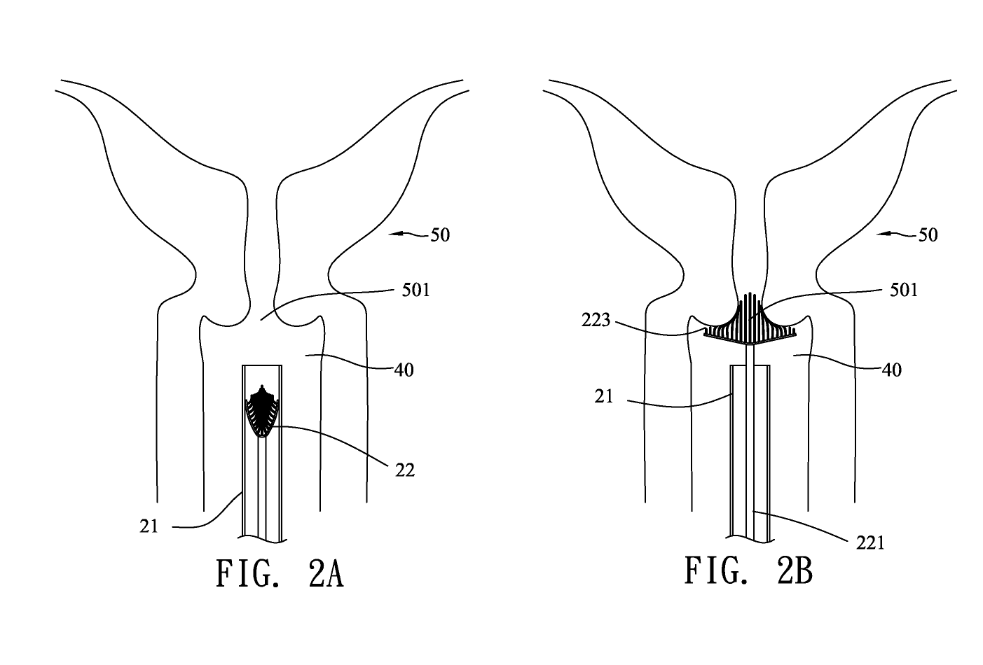

Referring to the drawings, and particularly to FIGS. 1 and 2, there is illustrated a foldable brush self-sampling device of the present invention. The foldable brush self-sampling device is easily used and manipulated for collecting cell-specimens from cervix, endometrium, rectum, nasal cavity and fauces, for diagnostic purposes to prevent or treat early cervical cancer, endometrial adenocarcinoma, carcinoma of the rectum and nasopharyngeal cancer.

Referring to FIG. 1A, the foldable brush self-sampling device basically includes a sheath 21 and a brush bar 22. The sheath 21 is a tube, such as a “telescoping” cylindrical tampon-like cardboard sheath, which houses the brush bar 22. The brush bar 22 includes an elongated stem 221 having two opposite end portions, a base 222 mounted on a front end portion of the stem 221 and a plurality of brush elements 223 formed on the base 221. The configurations of the brush elements 223 facilitate collecting cell specimens from a body cavity, such as cervix, endometrium, rectum, nasal cavity and fauces.

Take the cervix for example as follows, referring to FIG. 2B, the brush elements 223 placed in a central portion of base 222 are longer than the brush elements 223 placed in a periphery of the base 222 to facilitate collecting cervical cell specimens. The area of the base 222 is greater than the area of the cross-section of the sheath 21 to allow more brush elements 223 mounted thereon. And the base 222 is made of elastic material, such as plastic. When pushing the stem 221 back to the sheath 21, the base 222 is against the top end portion of the sheath 21 and is deformed (see FIG. 1B). As can be seen in FIG. 1C, the base 222 is folded inside the sheath 21 together with the brush elements 223.

When using, referring to FIG. 2A, in normal condition the brush bar 22 is folded in the sheath 21 and puts into the vagina 40 of a female by herself. Due to the outer diameter of the sheath 21 is smaller than the diameter of the vagina 40(also smaller than the width of man penis, maybe substantial the same with a finger), the foldable brush self-sampling device can insert into the vagina 40 easily. Then, referring to FIG. 2B, the stem 221 is pushed and the base 222 and the brush elements 223 would be unfolded. After insertion of the sheath 20 and push of the stem 221, the stem 221 is rotated to obtain the specimen from the cervix 501 of the uterus 50 and then retracted in order to return the sheath 21. The sheath 20 is made of solider material than the base 222 to assure that the top end portion of the sheath 21 does not collapse prior to the brush bar 22 returning. Because the configurations of the brush elements 223 facilitate collecting the cervix 501 cell specimens, the brush elements 223 will be positioned automatically to avoid collecting wrong cell specimens. The base 222 may be designed either plane or circular. The circular base 222 allows more brush elements 223 placed thereon, and the plane base 222 would be folded into sheath 21 easier. However, due to collect cell specimens by rotation, the plane base 222 would collect enough cell specimens for diagnostic purposes.

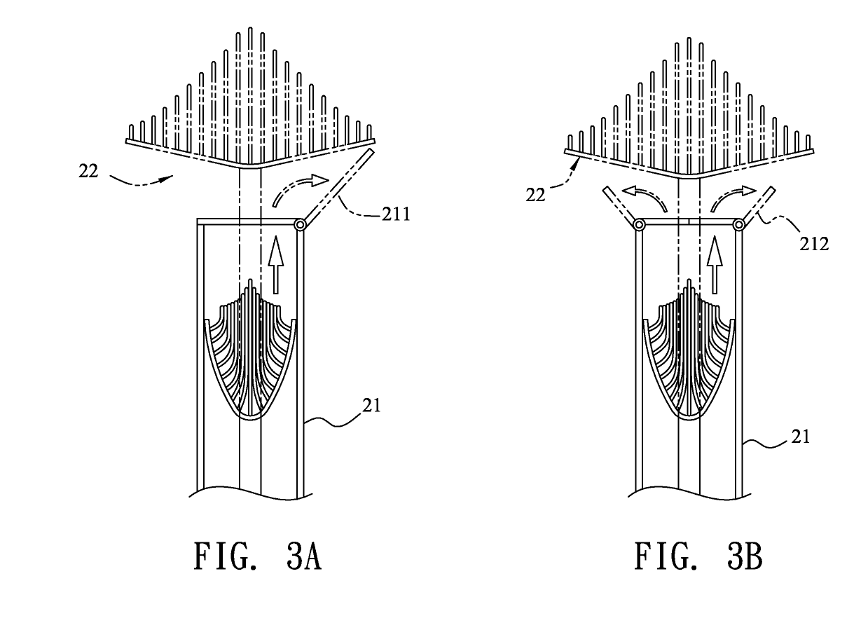

Also during insertion the vagina 40, for preventing to be stained with body fluid, such as leukorrhea, the device further includes a cover 211 in a front end of the sheath 21. Referring to FIGS. 3A and 3B, the cover 211 is closed normally. Before sampling, the brush bar 22 is pushed to open the cover 211 to allow the brush bar 22 expose the sheath 21. The cover 211 may be connected to the sheath 21 by a hinge (see FIG. 3A), or may include multiple caps 212, each connected to the sheath by a hinge, to make open easier.

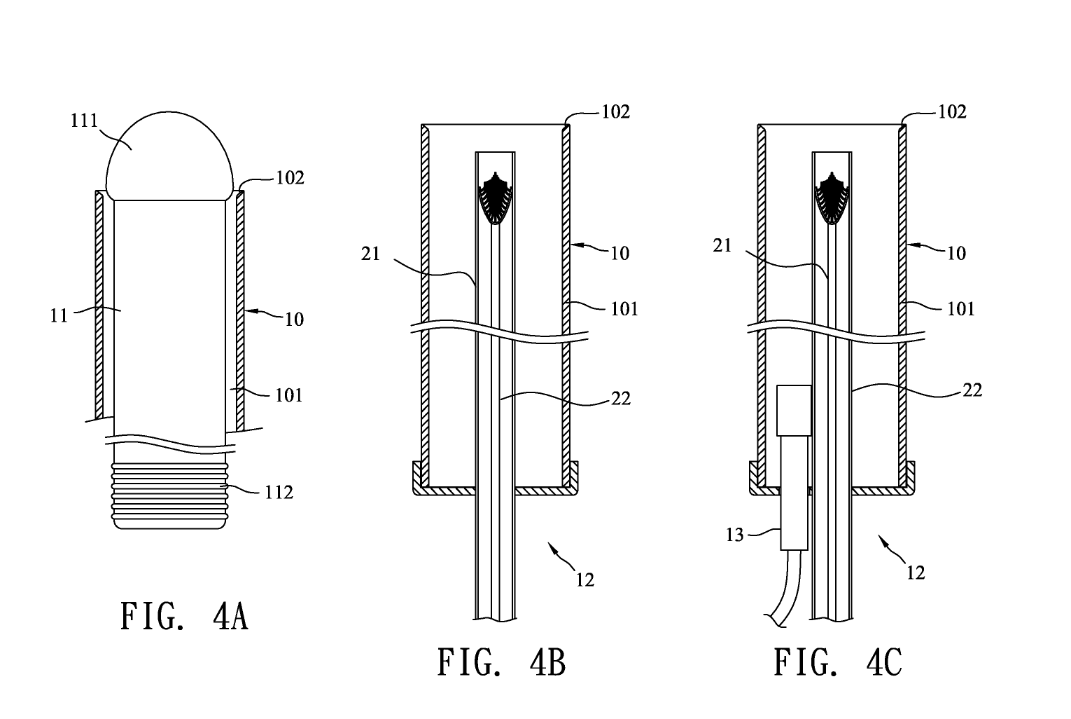

Referring to FIGS. 4A and 4B, for assuring the accuracy of collection, the foldable brush self-sampling device in accordance with the present invention further includes an outer shell 10 and a guide assembly 11. The outer shell 10 is tubular and has a capacity 101 therein for selectively assembling with the guide assembly 11 (see FIG. 4A) or the sampling assembly 12 (see FIG. 4B), including the sheath 21 and the brush bar 22. The outer shell 10 has an outer diameter that is almost the same as an inner diameter of woman vagina and preferably the outer diameter thereof is variety dependent on nature difference and different childbirth experiment from women. The outer shell 10 has a position unit 102 formed in the distal end for positing the outer shell 10 to a correct site, and preferably a wall of the outer shell 10 near the distal end is narrow gradually. More preferably, the wall of the outer shell 10 near the distal end has a bevel design for preventing from injuring vagina. On the other hand, the foldable brush self-sampling device is used in endometrium, rectum, nasal cavity and fauces by the same way. However, when using in endometrium, it may also use a vaginal dilator to hold open the vagina for collecting cell specimens easily.

The guide assembly 11 is selectively mounted in the capacity 101 of the outer shell 10 and has a proximal end, a distal end and a smooth head 111 formed on the proximal end. The head 111 can be hollow or solid cylinder for facilitating users insert the collector into the vagina. Preferably, the head 111 can be similar to man glans penis or a smooth curve or an arc-shape. A largest diameter of the head 111 is almost the same as an inner diameter of the capacity 101 of the outer shell 10 so that the guide assembly 11 will not depart from the outer shell 10 when the guide assembly 11 mounted in the outer shell 10 and a front part of the head 111 can expose from the outer shell 10 for guiding the collector into the vagina. Furthermore, the guide assembly 11 preferably has a skidproof portion 112 formed on the distal end thereof.

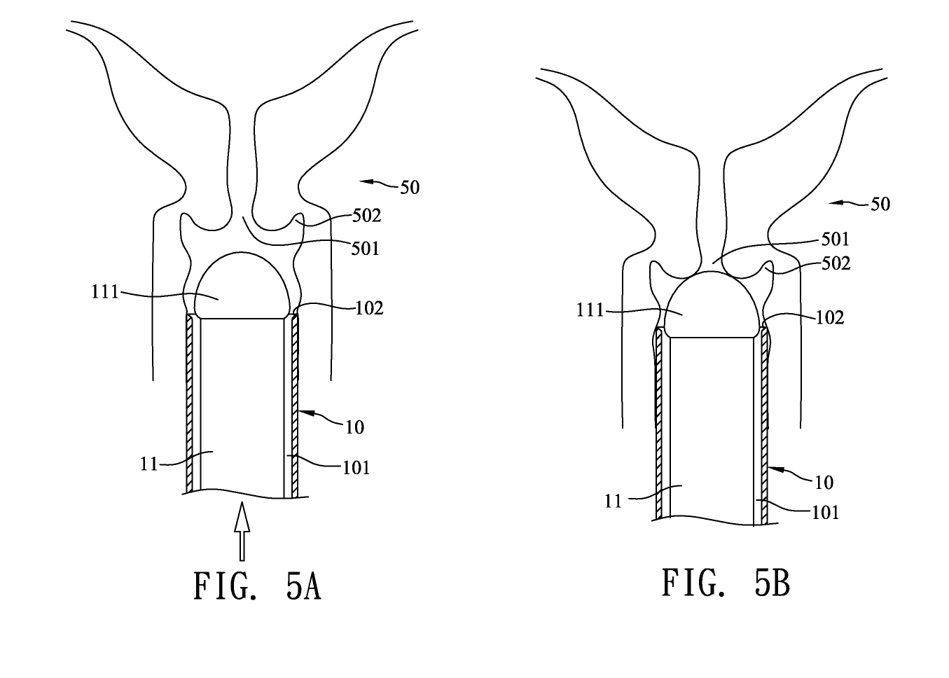

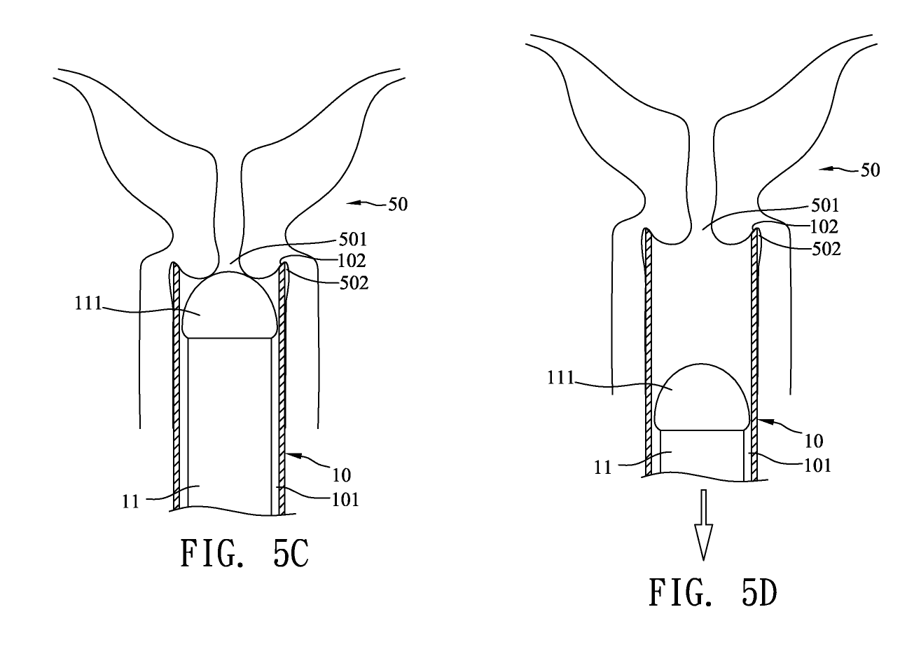

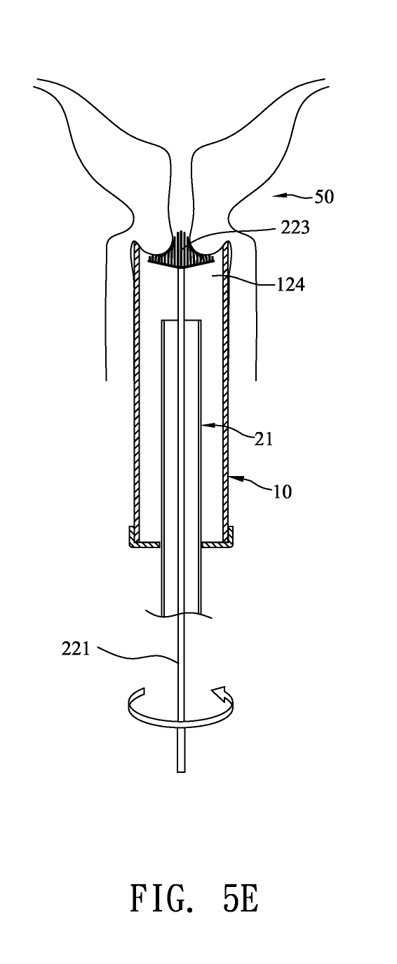

When using, the guide assembly 11 mounted in the outer shell 10 and the head 111 exposing from the distal end of the outer shell 10. The user can easily put the guide assembly 11 with the outer shell 10 into the vagina guiding by the smooth head 111 (see FIG. 5A) until the head 111 arriving and touching to the cervix 501 (see FIG. 5B). Because of the smooth design of the head 111, the guide assembly 11 not only guides the collector into the vagina but also protect the vagina, uterus 50, and cervix 501 from injuring. Then, the outer shell 10 continues to go inside until the position unit 102 on the distal end of outer shell 10 locking with a peripheral 502 of the cervix (see FIG. 5C). That is the point why users can collect cervical cells by themselves and not injury vagina or cervix 501 or collect wrong cells.

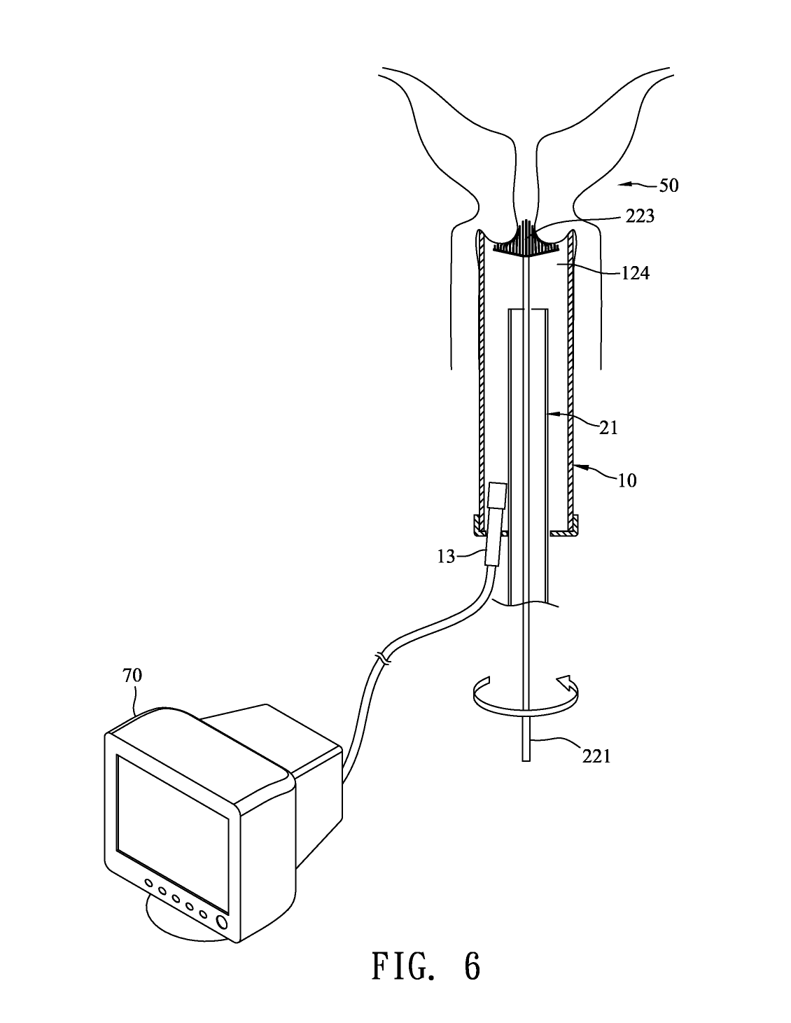

Following, the guide assembly 11 is taken out (see FIG. 5D) and the sampling assembly 12 is mounted (see FIG. 5E). Turning the stem 221 makes the brush elements 223 collect cells. With further reference to FIG. 1C, the foldable brush self-sampling device in accordance with the present invention further includes a camera 13 mounted in the sampling assembly 12 for facilitating users use. During collecting, referring to FIG. 6, the camera 13 connects to a display unit 700 for observing status of collection and users can adjust collection direction of the brush bar 22 at the same time such that an objective of indeed collecting is achieved. Through the camera 13 to show figures, users are more convenient to collect cervical cells. Of course, the camera 13 can connect with a lighting unit or a colposcopy for helping collecting.

While an illustrative and presently preferred embodiment of the invention has been described in detail herein, it is to be understood that the inventive concepts may be otherwise variously embodied and employed and that the appended claims are intended to be construed to include such variations except insofar as limited by the prior art.

What is claimed is:

1. A foldable brush self-sampling device, comprising:
a sheath; and
a brush bar mounted in the sheath and comprising a elongated stem and a plurality of brush elements formed in a front end portion of the stem by a base, the base is greater than the sheath and would be folded into the sheath together with the brush elements, wherein in using, the base is exposed from the sheath by pushing the stem, and the base would extend to permit collecting interior specimen of humans.

2. The foldable brush self-sampling device of claim 1, wherein the base is made of plastic.

3. The foldable brush self-sampling device of claim 1, wherein the brush elements placed in a central portion of base are longer than the brush elements placed in a periphery of the base.

4. The foldable brush self-sampling device of claim 1, wherein the sheath further comprises a cover in a front end thereof.

5. The foldable brush self-sampling device of claim 6, wherein the cover is connected to the sheath by a hinge.

6. The foldable brush self-sampling device of claim 6, wherein the cover includes multiple caps, each connected to the sheath by a hinge.

7. A foldable brush sampling device, comprising:
a tubular outer shell comprising an outer diameter almost the same as woman vagina, a proximal end and a distal end that has a circular position unit formed therein;
a guide assembly selectively mounted in the outer shell and comprising a smooth head formed in a proximal end thereof and exposing from the outer shell; and
a sampling assembly selectively mounted in the outer shell and comprising:
a sheath; and
a brush bar mounted in the sheath and comprising a elongated stem and a plurality of brush elements formed in a front end portion of the stem by a base, the base is greater than the sheath and would be folded into the sheath together with the brush elements, wherein in using, the base is exposed from the sheath by pushing the stem, and the base would extend-to permit collecting interior specimen of humans;
whereby, the guide assembly mounted in the outer shell guides the personal cervical cell collector to insert into the vagina guided by the head, and the head touches the woman cervix and the position unit locks with a peripheral of the cervix for achieving position effect, continuously, the guide assembly is taken out and the sampling assembly is put into the outer shell to sample.

8. The foldable brush self-sampling device of claim 7, wherein the base is made of plastic.

9. The foldable brush self-sampling device of claim 7, wherein the head of the guide assembly has an outer diameter substantial the same as an inner diameter of the outer shell.

10. The foldable brush self-sampling device of claim 9, wherein the head is a man glans penis shape.

11. The foldable brush self-sampling device of claim 7, wherein a wall near the distal end of the outer shell is narrow gradually to form the position unit.

12. The foldable brush self-sampling device of claim 11, wherein a front end of the position unit has a bevel design for protecting the cervix from injuring.

13. The foldable brush self-sampling device of claim 7, wherein the brush elements placed in a central portion of base are longer than the brush elements placed in a periphery of the base.

14. The foldable brush self-sampling device of claim 7, further comprising a camera for observing a status of collection.
